(19) United States
(12) Patent Application Publication
(10) Pub. No.: US 2009/0304659 A1
(43) Pub. Date: Dec. 10, 2009

(54) ANTI-CD8 ANTIBODIES BLOCK PRIMING OF CYTOTOXIC EFFECTORS AND LEAD TO GENERATION OF REGULATORY CD8+ T CELLS

(75) Inventors: Jacques F. Banchereau, Dallas, TX (US); Eynav Klechevsky, Haifa (IL); Anna Karolina Palucka, Dallas, TX (US)

Correspondence Address: CHALKER FLORES, LLP, 2711 LBJ FRWY, Suite 1036, DALLAS, TX, 75234, US

(73) Assignee: Baylor Research Institute, Dallas, TX (US)

(21) Appl. No.: 12/479,349

(22) Filed: Jun. 5, 2009

Related U.S. Application Data
(60) Provisional application No. 61/059,647, filed on Jun. 6, 2008.

(51) Int. Cl. A61K 35/12; C12N 5/00; A61P 37/06

(52) U.S. Cl. 424/93.71; 435/377

(57) ABSTRACT

The present invention includes compositions and methods for inducing tolerance in a subject in need thereof comprising providing the subject with an effective amount of an anti-CD8 antibody sufficient in induce CD8⁺ T cell immune tolerance to allogeneic antigens.

### CROSS-REFERENCE TO RELATED APPLICATIONS

This application claims priority to U.S. Provisional Application Ser. No. 61/059,647, filed Jun. 6, 2008, the contents of which is incorporated by reference herein in its entirety.

### TECHNICAL FIELD OF THE INVENTION

The present invention relates in general to the field of regulatory T cells, and more particularly, to compositions and methods for making and using anti-CD8 antibodies.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with immune cell tolerance.

U.S. Pat. No. 5,593,677 issued to Reichert, et al., teaches a method for prevention of graft versus host disease. The method includes a treatment and prevention of graft versus host disease in man through the combined use of anti-CD8 monoclonal antibodies and a CD4⁺ cell inactivator. The method for prevention of or prophylaxis against GVHD in a patient to undergo a bone marrow transplant, where bone marrow of an allogeneic donor has been matched to the patient for HLA compatibility, comprising the steps of treating the bone marrow of the donor with one or more anti-CD8 monoclonal antibodies and complement in an amount sufficient to deplete T cytotoxic/suppressor cells to less than 1%, transplanting the treated bone marrow to the patient, and administering to the patient an effective amount of Cyclosporine A sufficient to inactivate CD4⁺ cells.

U.S. Pat. No. 5,601,828 issued to Tykocinski, et al., relates to CD8 derivatives and methods of use for cellular modulation and enhancement of cellular engraftment. Specific and nonspecific immunomodulation, enhancement of cellular engraftment, and modulation of nonimmune cells are achieved by using various membrane-binding and soluble CD8 compositions. In this patent, the method for specifically reducing T-cell proliferation or cytotoxicity directed to an alloantigen or a MHC-associated antigen, includes providing a non-naturally occurring membrane which presents in, or on its surface, an extracellular domain portion of CD8 and the alloantigen or the MHC-associated antigen wherein the extracellular domain portion of CD8 comprising at least the Immunoglobulin V homolog domain is covalently linked to a molecule which binds covalently or non-covalently with a cell surface molecule, and exposing the membrane to T-cells able to respond to the alloantigen or MHC-associated antigen, for a time and under conditions sufficient to reduce the specific cellular immune response of the T-cells to the alloantigen or MHC-associated antigen.

U.S. Pat. No. 5,876,708, issued to Sachs, relates to Allogeneic and xenogeneic transplantation and methods for inducing tolerance including administering to the recipient a short course of help reducing treatment or administering a short course and methods of prolonging the acceptance of a graft by administering a short course of an immunosuppressant. The method includes inducing tolerance in a recipient primate of a first species to a graft obtained from a mammal of a second species by introducing into the recipient, hematopoietic stem cells of the second species, implanting the graft in the recipient; inactivating T cells of the recipient; and, administering to the recipient a short course of an immunosuppressive agent, wherein the agent is not an anti-T cell antibody and the short course is equal to or less than 120 days, thereby inducing tolerance to the graft.

U.S. Pat. No. 6,911,220, also issued to Sachs relates to allogeneic and xenogeneic transplantation. The invention provides methods for restoring or inducing immunocompetence, the methods including the step of introducing donor thymic tissue into the recipient. The invention also provides methods for inducing tolerance in a recipient including introducing donor thymic tissue into the recipient. The invention further provides methods of inducing tolerance including administering to the recipient a short course of help reducing treatment or administering a short course and methods of prolonging the acceptance of a graft by administering a short course of an immunosuppressant.

United States Patent Application No. 20070166307, filed by Bushell, et al., is directed to suppression of transplant rejection. Briefly, a transplant rejection in an animal suppressed by administration of an antibody directed at a cell surface antigen selected from the group consisting of CD4, CD8, CD154, LFA-1, CD80, CD86 and ICAM-1, preferably an anti-CD4 antibody, together with a non-cellular protein antigen to generate in the animal a population of regulatory T-lymphocytes; reactivating the population of regulatory T-lymphocytes by further administration to the animal of the non-cellular protein antigen; and transplanting the organ or tissue whilst the population of regulatory T-lymphocytes is activated is taught. Regulatory T cells can be generated ex vivo by culturing T cells with an antibody directed at a cell surface antigen selected from the group consisting of CD4, CD8, CD154, LFA-1, CD80, CD86 and ICAM-1, in the presence of cells that present either alloantigen or a non-cellular protein antigen. Ex vivo generated T-lymphocytes can be used as an alternative method of overcoming transplant rejection or in combination with the in vivo method. A similar approach can be adopted for the treatment of autoimmune conditions.

United States Patent Application No. 20050042217, filed by Qi, et al., for a specific inhibition of allorejection. The specification provides methods and compositions for specifically inhibiting both cellular and humoral immune responses to alloantigen, thereby finding use in extending the survival of transplant allografts and treating graft versus host disease in transplant recipients. The method teaches inhibiting a host immune response to target cell-specific antigens, by contacting a target cell expressing the antigen with an expression vector encoding a CD8 polypeptide with the CD8 a-chain, wherein the CD8 polypeptide is expressed by the target cell and whereby a host immune response against the target cell is specifically inhibited. That is, an increase in CD8 on the target cell specifically inhibits the immune response.

### SUMMARY OF THE INVENTION

The present invention includes compositions and methods for inducing immune tolerance in a subject in need thereof In one embodiment the compositions and methods may be used to induce immune tolerance in a subject by providing the subject with an effective amount of an anti-CD8 antibody sufficient in induce CD8+ T cell immune tolerance to antigens. In one aspect, the anti-CD8 antibody is humanized. In another aspect, the anti-CD8 antibody is non-depleting. The method may also include the generation of suppressor T cells as determined by measuring or determining one or more of the following phenotypes: a reduction in granzyme A, a reduction in granzyme B, a reduction of perforin, secretion of reduced amounts of IL-2, IFN-γ or both, secretion of IL-10 or a combinations thereof. In one aspect, the generation of suppressor T cells is the proliferation of suppressor T cells that secrete IL-10. In another aspect, the anti-CD8 antibody is selected from cM-T807, T8, RPA-T8, HIT8a, Leu 2, T8, and OKT8. In one example, the antigen is allogeneic.

In another embodiment, the present invention includes compositions and methods to reduce transplant rejection in a transplant patient while maintaining other immune responses by treating isolated CD8+ T cells with an amount of anti-CD8 non-depleting, blocking antibody effective to trigger the generation of suppressor CD8+ T cells characterized by one or more of the following phenotypes: a reduction in granzyme A, a reduction in granzyme B, a reduction of perforin, secretion of reduced amounts of IL-2, IFN-γ or both, secretion of IL-10 or a combinations thereof; and introducing the suppressor CD8+T cells into the transplant patient. In one aspect, the CD8+ T cells are incubated with isolated dendritic cells obtained from monocytes cultured with GM-CSF and IFN-α-2b (IFN-DCs). In another aspect, the dendritic cells are Langerhans cells (LCs) generated in-vitro by culturing CD34+ human peripheral cells for nine to ten days with GM-CSF, Flt3-L and TNFα. Another example of dendritic cells are CD1a+CD14− LCs. In another aspect, the anti-CD8 antibody down-regulates the immune response to the engrafted organ without affecting the immune response to viruses. In another aspect, the CD8+ T cells treated with the anti-CD8 antibody are high-avidity, antigen-specific naïve T cells. In one non-limiting example, the anti-CD8 antibody are selected from cM-T807, T8, RPA-T8, HIT8a, Leu 2, T8, OKT8 and the anti-CD8 antibodies listed in Table 1. In one aspect of a treatment for T cells in vitro, the anti-CD8 antibody is provided in a CD8+ T cell culture at between 0.5 to 5,000 ng/ml. For in vivo use, the present invention may be provided to achieve similar levels on an equivalent concentration in blood depending on the weight of the individual.

In another aspect, the present invention may also include the steps of isolating peripheral blood mononuclear cells, isolating LC precursors from the peripheral blood mononuclear cells, culturing the LC precursors with GM-CSF, Flt3-L and TNFα to make LCs, isolating T cells from peripheral blood mononuclear cells and co-culturing the LCs and the T cells in the presence of an anti-CD8 antibody under conditions that generate suppressor T cells, and reintroducing the T cells, the LCs or both into a patient prior to, in conjunction with or after transplantation. In another aspect, the method may also include the steps of isolating peripheral blood mononuclear cells from the transplant patient, isolating LCs and culturing the LCs GM-CSF, Flt3-L and TNFα, isolating T cells from the transplant patient and co-culturing the LCs and the T cells in the presence of an anti-CD8 antibody to generate suppressor T cells, and reintroducing the T cells, the LCS or both into the patient prior to, in conjunction with or after transplantation. In one aspect, the suppressor CD8+ T cells have an increased expression of type 2 cytokines (IL-4, IL-5 and IL-13) and IL-10.

Yet another embodiment of the present invention includes method of making suppressor T cells and the cells made thereby, the method including isolating peripheral blood mononuclear cells, isolating LC precursors from the peripheral blood mononuclear cells, culturing the LC precursors with GM-CSF, Flt3-L and TNFα to make LCs, isolating T cells from peripheral blood mononuclear cells and co-culturing the LCs and the T cells in the presence of an anti-CD8 antibody under conditions that generate suppressor T cells. In one aspect, the anti-CD8 antibody down-regulates the immune response to the engrafted organ without affecting the immune response to viruses. In one aspect, the CD8+ T cells are high-avidity antigen-specific naïve T cells. In one aspect, the Langerhans cells are CD1a+CD14− LCs. In another aspect, the CD1a+CD14− Langerhans cells are obtained by cell sorting. In yet another aspect, the Langerhans cells are generated in-vitro by culturing for nine to ten days CD34+ HPCs with GM-CSF, Flt3-L and TNFα. In one aspect, the anti-CD8 antibody is selected from cM-T807, T8, RPA-T8, HIT8a, Leu 2, T8, and OKT8. The anti-CD8 antibody may also be provided in the culture at between 0.5 to 5,000 ng/ml.

In yet another embodiment, the present invention includes a method of making suppressor T cells, and the suppressor T cells made thereby, by isolating peripheral blood mononuclear cells, isolating monocytes from the peripheral blood mononuclear cells, culturing the monocytes with GM-CSF and IFN-α-2b to make (IFN-DCs), isolating T cells from peripheral blood mononuclear cells and co-culturing the IFN-DCs and the T cells in the presence of an anti-CD8 antibody under conditions that generate suppressor T cells.

Another embodiment of the present invention is a method for affecting an immune response, by administering a composition that includes suppressor T cells made by isolating peripheral blood mononuclear cells, isolating LC precursors from the peripheral blood mononuclear cells, culturing the LC precursors with GM-CSF, Flt3-L and TNFα to make LCs, isolating T cells from peripheral blood mononuclear cells and co-culturing the LCs and the T cells in the presence of an anti-CD8 antibody under conditions that generate the suppressor T cells.

Yet another embodiment of the present invention is a method of inhibiting rejection of a transplanted tissue in a mammal by introducing a suppressor T cell made by a method comprising isolating peripheral blood mononuclear cells, isolating LC precursors from the peripheral blood mononuclear cells, culturing the LC precursors with GM-CSF, Flt3-L and TNFα to make LCs, isolating T cells from peripheral blood mononuclear cells and co-culturing the LCs and the T cells in the presence of an anti-CD8 antibody under conditions that generate the suppressor T cells.

In another embodiment, the present invention is a composition that reduces transplant rejection that includes an effective amount of suppressor T cells sufficient to reduce transplant rejection without eliminating other immune responses, wherein the suppressor T cells are generated from isolated peripheral blood T cells co-cultured with mature LCs in the presence of an anti-CD8 antibody under conditions that generate the suppressor T cells. In one aspect, the anti-CD8 antibody is selected from cM-T807, T8, RPA-T8, HIT8a, Leu 2, T8, and OKT8. In another aspect, the anti-CD8 antibody is provided in the culture at between 0.5 to 5,000 ng/ml. In one aspect, the cells are frozen and resuspended in a medium for injection prior to use.

### BRIEF DESCRIPTION OF THE DRAWINGS

For a more complete understanding of the features and advantages of the present invention, reference is now made to the detailed description of the invention along with the accompanying figures and in which:

FIGS. 1a to 1c increased CD8 expression is induced on LCs-primed CD8+ T cells but not on IntDCs primed CD8+ T cells. FIG. 1a shows a flow cytometry analysis of CD8 expression level on naïve CD8+ T cells primed by CD34-DCs subsets. CD8 on LCs primed CD8+ T cells (black line); CD8 on IntDCs primed CD8+ T cells (grey line). FIG. 1b are naïve Mart-1 specific CD8+ T cells primed by LCs express higher level of CD8 compare to IntDCs-primed Mart-1 specific naïve CD8+ T cells. FIG. 1c shows memory Flu-MP specific CD8+ T cells activated by both subsets, i.e., LCs or IntDCs, express equal levels of surface CD8.

FIGS. 2a through 2h shows the role of CD8 in DCs-mediated autologous naïve CD8+ T cell priming. FIG. 2a shows autologous Mart-1 specific CD8+ T cells priming is dependent on CD8 ligation. FIG. 2b shows the percentage of Mart-i specific CD8+ T cells measured during priming with LCs between days 1 to 9. FIG. 2c shows 3 different clones in at lease 3 independent experiments with at least 3 different donors, showed a significant blockage of naïve allogeneic proliferation induced by LCs. T8 Beckman upper panel, RPA-T8 middle panel, OKT8 lower panel. FIG. 2d shows anti-CD8 blocks priming of autologous naïve CD8+ T cells in a dose dependent fashion. IC50 as determined at 50 ng/ml. FIG. 2e shows the percentage of Mart-1 specific CD8 T cells, anti-CD8 efficiently block antigen specific CD8 T cells priming even when added as late as 70 h after co-culture initiation. FIG. 2f shows MART-1 specific CD8+ T cells, Primed by peptide loaded LCs in the presence of low dose of anti-CD8 Mab stain tetramer with lower intensity compared to antigen specific CD8+ T cells primed in the presence of isotype control. FIG. 2g shows the Correlation between the tetramer intensity to the dose of anti-CD8 Mab used. FIG. 2h. Priming of MART-1 specific was blocked by anti-CD8 even when the DCs were loaded with high concentration of peptide 100 uM or when the peptide was presence throughout the culture (left panel); right panel: number of MART-1 specific CD8⁺ T cells primed by IFN-DCs and loaded with the indicated peptide concentrations. FIG. 2i shows the anti-CD8 block priming of MART-1 (upper panel) or gp100 (lower panel) specific CD8+ T cells by IFN-DCs

FIGS. 3a through 3g shows that CD8 ligation is critical for allogeneic naïve CD8+ T cells priming. FIG. 3a shows Naïve CD8+ T cells proliferation in response to allogeneic DCs in the presence of anti-CD8 or Isotype control was determined by cellular thymidine incorporation. FIG. 3b shows naïve T cells proliferation in response to allogeneic LCs in the presence of anti-CD8 or Isotype control was determined by CFSE dilution. CD8+ T cells in the upper panel and naïve CD4+ T cells proliferation in lower panel. FIG. 3c shows the dose titration of 30 ng/ml to 3 ug/ml anti-CD8 showed maximal inhibition of CD8 T cell proliferation at 30 ng/ml (upper panel). No inhibition of CD4+ T cell proliferation was detected in any concentration of anti-CD8 Mab used (lower panel). FIGS. 3d and 3e show anti-CD8 Mab prevents alloproliferation of naïve CD8+ T cells stimulated by skin derived DCs, epidermal LCs (3d) or dermal DCs (3e) 50% inhibition was detected at 30 ng/ml. FIGS. 3f and 3g show peptide-loaded LCs and naïve CD8+ T cells create clusters which are apparent on day 9 of the co-culture (3g), while in the presence of anti-CD8, clusters formation is inhibited (3f). magnitude 20× upper panel 40× lower panel.

FIGS. 4a through 4f shows that anti-CD8 does not block secondary CD8+ T cells responses against viral or allogeneic antigens. FIG. 4a shows the frequency of FluMP-specific CD8+ T cells analyzed with FluMP-HLA-A201 tetramer 9 days after activation with FluMP peptide-loaded LCs from an HLA-A201 donor in the presence of 3 μg/ml anti-CD8 Mab (left panel) or Isotype matched control (right panel). FIG. 4b shows that anti-CD8 Mab does not block LCs induced secondary Flu-Mp specific response at any concentration of Mab used, as analysed by Flu-MP-HLA-A201 tetramer. FIG. 4c shows the frequency of FluMP-specific CD8+ T cells analyzed with FluMP-HLA-A201 tetramer 9 days after activation with FluMP peptide-loaded IntDCs from an HLA-A201 donor in the presence of 3 μg/ml anti-CD8 Mab (left panel) or Isotype matched control (right panel). FIG. 4d shows that anti-CD8 Mab does not block IntDCs induced secondary Flu-Mp specific response at any concentration of Mab used, as analysed by Flu-MP-HLA-A201 tetramer. FIG. 4e shows the lack of inhibition by anti-CD8 is not limited to a particular anti-CD8 clone as 2 different clones; T8 beckman (left panel) and RPA-T8 (right panel) showed no inhibition of Flu-MP specific CD8+ T cells proliferation induced by peptide loaded LCs after 9 days of culture in the presence 3 ug/ml of the indicated anti-CD8 clone or the Isotype matched control. FIG. 5f shows the memory response against allogeneic antigen is not blocked by anti-CD8. Thymidine incorporation of a secondary allogeneic co-culture shows that allogeneic LCs (left panel) or IntDCs (right panel), were effective at inducing allospecific secondary response whether anti-CD8 Mab or isotype matched control were presence in the culture.

FIGS. 5a and 5b show a functional analysis of CD8+ T cells primed in the presence of anti-CD8 mAb. In FIG. 5a allogeneic naïve CD8+ T cells primed in the presence of anti-CD8 mAb were analyzed after 6 d by flow cytometry for the expression of activation and effector molecules. In FIG. 5b allogeneic naïve CD8+ T cells primed in the presence of anti-CD8 Mab secrete Type 2 and regulatory cytokines. Naïve CD8+ T cells were cultured over LCs in the presence or absence of anti-CD8. After 6 d, the proliferated (CFSElow) cells were sorted and restimulated for 24 h with anti-CD3 and anti-CD28 beads and IFN-γ, IL-2-, IL-4, IL-5, IL-10, and IL-13 were measured in luminex, multiplex bead assay. Data presented are from 3 independent studies.

FIGS. 6a and 6b show that CD8+ T cells primed in the presence of anti-CD8 are suppressors T cells. FIG. 6a shows the capacity of primed T cells to suppress primary T cell responses was tested by stimulating naive CD8+ T cells with allogeneic DCs in the presence of decreasing numbers of syngeneic T cells primed by in vitro LCs in the presence of anti-CD8 or isotype control. ³[H]thymidine incorporation was assessed after 6 d. Results are representative of three independent studies. FIG. 6b shows naive CD8 T cells (donor A) were stimulated with allogeneic LCs from donor B in the presence of CD8 Tr cells primed to in vitro LCs from donor C in the presence of anti-CD8 or Isotype control. Results are representative of three independent experiments

FIGS. 7a and 7b show the effect of anti-CD8 treatment prevents graft versus host in human-mouse model in vivo. FIG. 7a shows the results using humanized mice injected with allogeneic CD8+ T cells and anti-CD8 MAb or isotype control. In one out of two studies, anti-CD40 was injected to induce activation. Mice treated with isotype control antibodies developed clinical symptoms of chronic graft versus host disease, with rush around the eye (shown), weight loss and weakness, while mice treated with anti-CD8 did not. FIG. 7b shows the results from mice were harvested and the CD8⁺ T cells from BM and blood were analyzed for the expression of activation markers CD25 and CD103.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Dendritic cells (DCs) are potent APCs responsible for inducing Ag-specific immunity¹. Several populations of DCs exist that take up residence in different tissues, and have distinct functional attributes¹. The healthy skin hosts at least two DCs populations Langerhans cell (LCs) in the epidermis and Interstitial DCs in the dermis. These DCs migrate into the draining lymphoid organs for peripheral tolerance when unactivated and immunity when activated. Other DCs are found residing in secondary lymphoid organs and circulating in the blood. Much progress in the understanding of DC biology came from the studies performed with DCs generated in vitro. In particular, the culture of CD34+ hematopoietic progenitor cells (HPCs) in the presence of TNFα and GM-CSF give rise to both Interstitial DCs and Langerhans cells². The present inventors have shown that LCs but not IntDCs are particularly efficient in priming naïve CD8+ T cells. Also, both subsets are equally efficient at inducing a memory response and CD8+ T cells activated by both subsets show equal expression of CD8 molecule.

CD8 is a surface glycoprotein that functions as a coreceptor for TCR recognition of peptide antigen complexed with MHC Class I molecule (pMHCI). It is expressed either as an αα homodimer or as an αβ heterodimer³, both chains expressing a single extracellular Ig superfamily (IgSF) V domain, a membrane proximal hinge region, a transmembrane domain, and a cytoplasmic tail³. CD8 interacts with β₂m and the β2 and α3 domains of MHC Class I molecules using its β strands and the complementary determining regions (CDRs) within the extracellular IgSF V domain. This association increases the adhesion/avidity of the T cell receptor with its Class I target. In addition, an internal signaling cascade mediated by the CD8α chain associated tyrosine protein kinase p56lck^{4,5} leads to T cell activation. Lck is required for activation and expansion of naive CD8+ T cells; however its expression is not essential for responses of memory CD8+ T cells to secondary antigenic stimulation in vivo or in vitro^{6,7}. As shown by either CD8α or CD8β gene targeted mice, CD8 plays an important role in the maturation and function of MHC Class I-restricted T lymphocytes^{8,9}. One patient suffering from repeated bacterial infections was found to display a CD8 deficiency due to a single mutation in the CD8α gene. The lack of CD8 did not appear to be essential for either CD8⁺ T cell lineage commitment or peripheral cytolytic function¹⁰.

Any of a number of well-known anti-CD8 antibodies, including monoclonal antibodies, may be used in conjunction with the present invention, such as those that are part of the International Workshops on Human Leucocyte Differentiation Antigens (HLDA), including: 2D2; 4D12.1; 7B12 1G11; 8E-1.7; 8G5; 14; 21Thy; 51.1; 66.2; 109-2D4; 138-17; 143-44; 278F24; 302F27; AICD8.1; anti-T8; B9.1.1; B9.2.4; B9.3.1; B9.4.1; B9.7.6; B9.8.6; B9.11; B9.11.10; BE48; BL15; BL-TS8; BMAC8; BU88; BW135/80; C1-11G3; C10; C12/D3; CD8-4C9; CLB-T8/1; CTAG-CD8, 3B5; F80-1D4D11; F101-87 (S-T8a); G10-1; G10-1.1; HI208; HI209; HI212; HIT8a; HIT8b; HIT8d; ICO-31; ICO-122; IP48; ITI-5C2; ITM8-1; JML-H7; JML-H8; L2; L533; Leu-2a; LT8; LY17.2E7; LY19.3B2; M236; M-T122; M-T415; M-T805; M-T806; M-T807; M-T808; M-T809; M-T1014; MCD8; MEM-31; MEM-146; NU-Ts/c; OKT8; OKT8f, P218; RPA-T8; SM4; T8; T8 /2T8-19; T8 /2T8-2A1; T8 /2T8-1B5; T8 /2T8-1C1; T8 /7Pt3F9; T8 /21thy2D3; T8 /21thy; T8 /TPE3FP; T8b; T41D8; T811; Tü68, Tü102; UCHT4; VIT8; VIT8b; WuT8-1; X107; YTC141.1; and/or YTC182.20.

TABLE 1
Examples of anti-CD8 antibodies may include those commercially available
such as those from Santa Cruz Biotechnology, Inc., and include one
or more of the following, or humanized versions thereof:

| ANTIBODY | ISOTYPE | EPITOPE | APPLICATIONS | SPECIES |
|---|---|---|---|---|
| CD8 (0.N.66) | mouse IgG₁ | C-terminus (h) | WB, IP, IF, IHC(P) | Human |
| CD8 (1.BB.720) | mouse IgG₁ | FL (rabbit) | IF, FCM | Rabbit |
| CD8 (12.C7) | mouse IgG₁ | FL (rabbit) | IF, FCM | Rabbit |
| CD8 (14) | mouse IgG₁ | FL (h) | IF | Human |
| CD8 (15-11C5) | mouse IgG₂ₐ | FL (r) | IF | Rat |
| CD8 (2.43) | rat IgG_{2b} | FL (m) | IF, FCM | Mouse |
| CD8 (32-M4) | mouse IgG₂ₐ | FL (h) | WB, IP, IF, FCM | Human |
| CD8 (38.65) | mouse IgG₂ₐ | FL (sheep) | IP, IF, FCM | sheep, cow |
| CD8 (5F10) | mouse IgG₁ | FL (h) | IF, IHC(P), FCM | Human |
| CD8 (5H10-1) | rat IgG_{2b} | FL (m) | IF, FCM | Human |
| CD8 (6A238) | mouse IgG₁ | N/A | FCM | Horse |
| CD8 (6A243) | rat IgG₁ | FL (dog) | FCM | human, dog |
| CD8 (6D17) | mouse IgG₂ₐ | FL (h) | IP, FCM | Human |
| CD8 (733) | mouse IgG₁ | N/A | FCM | Human |
| CD8 (8.F.36) | mouse IgG₁ | FL (h) | FCM | Human |
| CD8 (B-H7) | mouse IgG₁ | FL (h) | IF | Human |
| CD8 (B334) | mouse IgM | N/A | IF | Human |
| CD8 (C8/144B) | mouse IgG₁ | C-terminus (h) | WB, IP, IF, IHC(P) | Human |
| CD8 (CT6) | mouse IgG₁ | FL (guinea pig) | IF, FCM | guinea pig |
| CD8 (CVS8) | mouse IgG₁ | N/A | FCM | Horse |
| CD8 (DK25) | mouse IgG₁ | N/A | IF | Human |
| CD8 (fCD8) | mouse IgG₁ | N/A | IP, IF, FCM | Cat |
| CD8 (G28) | mouse IgG₂ₐ | FL (r) | IP, IF, FCM | Rat |
| CD8 (H030-1.2) | mouse IgM | N/A | IF | Human |
| CD8 (hCD8) | mouse IgG₂ₐ | FL (h) | FCM | Human |
| CD8 (HIT8a) | mouse IgG₁ | FL (h) | IF, FCM | Human |
| CD8 (ICO-31) | mouse IgG₁ | FL (h) | FCM | Human |
| CD8 (JXYT8) | rat IgM | FL (m) | IF, IHC(P) | Mouse |
| CD8 (LT8) | mouse IgG₁ | FL (h) | FCM | Human |
| CD8 (M211) | mouse IgG₁ | FL (h) | IP | Human |
| CD8 (M236) | mouse IgG₁ | FL (h) | IP | Human |
| CD8 (MCD8) | mouse IgG₁ | FL (h) | IF, IHC(P), FCM | Human |
| CD8 (MEM-31) | mouse IgG₂ₐ | FL (h) | IP, FCM | Human |
| CD8 (MEM-87) | mouse IgG₁ | FL (h) | IP, FCM | Human |
| CD8 (MIL-12) | mouse IgG₂ₐ | N/A | FCM | Pig |
| CD8 (RAVB3) | mouse IgG₁ | Fl (h) | WB, IF, FCM | Human |
| CD8 (RFT-8) | mouse IgG₁ | N/A | IF, FCM | Human |
| CD8 (RIV11) | mouse IgG₁ | FL (h) | IF, FCM | Human |
| CD8 (RPA-T8) | mouse IgG₁ | N/A | IF, FCM | Human |
| CD8 (UCH-T4) | mouse IgG₂ₐ | FL (h) | IP, IF, IHC(P), FCM | Human |
| CD8 (YCATE 55.9) | rat IgG₁ | FL (dog) | FCM | h, dog |
| CD8 (YTC 141.1HL) | rat IgG_{2b} | FL (h) | FCM | Human |
| CD8 (YTC 182.20) | rat IgG_{2b} | FL (h) | FCM | Human |
| CD8 (YTS 156.7.7) | rat IgG_{2b} | FL (m) | FCM | Mouse |
| CD8 (YTS169.4) | rat IgG_{2b} | N/A | IF, FCM | Mouse |
| CD8-α (76-2-11) | mouse IgG₂ₐ | N/A | IP, FCM | Pig |
| CD8-α (CT-8) | mouse IgG₁ | N/A | IP, IF, FCM | Chicken |
| CD8-α (EP72) | mouse IgG_{2b} | N/A | IP, IF, FCM | Chicken |
| CD8-α (143-44) | mouse IgG₁ | FL (h) | IF, FCM | Human |
| CD8-α (3-298) | mouse IgG_{2b} | N/A | IP, IF, FCM | Chicken |
| CD8-α (3H842) | rat IgG₂ₐ | FL (m) | IP, IF, FCM | Mouse |
| CD8-α (4j9) | mouse IgG₁ | N/A | IP, IF, FCM | Chicken |
| CD8-α (53-6.7) | rat IgG₂ₐ | FL (m) | IP, IF, FCM | Mouse |
| CD8-α (5J7) | mouse IgG₁ | FL (h) | IF, FCM | Human |
| CD8-α (5K100) | mouse IgG_{2b} | N/A | IP, IF, FCM | Chicken |
| CD8-α (5K97) | mouse IgG_{2b} | N/A | IP, IF, FCM | Chicken |
| CD8-α (6A242) | mouse IgG₁ | FL (r) | IP, IF, IHC(P), FCM | Rat |
| CD8-α (C-19) | goat IgG | C-terminus (h) | WB, IF | Human |
| CD8-α (CA9.JD3) | mouse IgG₂ₐ | FL (dog) | IP, IF, FCM | Dog |
| CD8-α (D-9) | mouse IgG₂ₐ | 22-182 (h) | WB, IP, IF, IHC(P) | m, r, h |
| CD8-α (H-160) | rabbit IgG | 22-182 (h) | WB, IP, IF | m, r, h |
| CD8-α (IBL-3/25) | rat IgG₁ | FL (m) | IP, IF, FCM | Mouse |
| CD8-α (KT15) | rat IgG₂ₐ | FL (m) | IF, FCM | Mouse |
| CD8-α (OX8) | mouse IgG₁ | FL (r) | IP, IF, IHC(P), FCM | Rat |
| CD8-α (R-15) | goat | C-terminus (r) | WB, IP, IF | m, r |
| CD8-α (YTS105.18) | rat IgG_{2b} | FL (m) | FCM | Mouse |
| CD8-α (YYEX) | mouse IgG_{2b} | extracellular (h) | FCM | Human |
| CD8-β (1.BB.574) | mouse IgG₂ₐ | FL (h) | FCM | Human |
| CD8-β (2ST8.5H7) | mouse IgG₂ₐ | FL (h) | FCM | Human |
| CD8-β (341) | mouse IgG₁ | FL (r) | WB, IP, FCM | Rat |
| CD8-β (3H901) | mouse IgG₂ₐ | FL (h) | FCM | Human |
| CD8-β (53-5.8) | rat IgG₁ | FL (m) | IP, IF, FCM | Mouse |
| CD8-β (5F2) | mouse IgG₁ | internal (r) | WB, IP, IF, IHC(P), FCM | Human |
| CD8-β (C-16) | goat IgG | C-terminal (h) | WB, IF | Human |
| CD8-β (EP42) | mouse IgG₂ₐ | N/A | IP, IF, FCM | Chicken |
| CD8-β (F-5) | mouse IgG₂ₐ | 22-170 (h) | WB, IP, IF, IHC(P) | Human |
| CD8-β (H-149) | rabbit IgG | 22-170 (h) | WB, IP, IF | m, r, h |
| CD8-β (H35-17.2) | rat IgG_{2b} | FL (m) | IP, IF, IHC(P), FCM | Mouse |
| CD8-β (M-20) | goat IgG | C-terminus (m) | WB, IP, IF | Mouse |
| CD8-β (R-20) | goat IgG | C-terminus (r) | WB, IF | Rat |
| CD8α/β (vpg 9) | mouse IgG₁ | FL (cat) | IF, FCM | Cat |

Non-limiting examples of humanized anti-CD8 antibodies include cM-T807 (Centocor, Mass.), and TRX2 (Oxford Therapeutic Antibody Centre, Oxford University, Oxford, United Kingdom).

Dendritic cells (DCs) initiate and polarize antigen-specific immune responses. Human myeloid DCs (mDCs) include distinct subsets such as Langerhans cells and interstitial (dermal) DCs that reside in human skin. We have reported that Langerhans cells when compared to Interstitial DCs are particularly powerful at priming naïve CD8+ T cells against allogenic and autologous antigens, whereas both mDCs subsets were equally efficient at inducing a secondary response. The current study was performed to analyze the parameters which might explain the superior functions of LCs in inducing CD8+ T cell priming. LCs primed CD8+ T cells express higher levels of CD8 compared to IntDCs primed CD8+ T cells, while antigen specific memory CD8+ T cells induced by both subsets, present equal levels of CD8.

It is shown herein that anti-CD8 monoclonal antibodies block DC-mediated in vitro priming of autologous as well as allogenic antigens CTLs. The CD8+ T cells primed in the presence of anti-CD8 failed to kill targets and produced type 2 (IL-4, IL-5, IL-13) and regulatory (IL-10) cytokines. Furthermore, the CD8⁺ T cells primed in the presence of anti-CD8 mAb were able to inhibit an alloreaction and thus acted as suppressor CD8+ T cells. However, induction of secondary CTL responses such as those to Influenza and CMV were not disturbed. Likewise anti-CD8 mAbs did not alter CD4+ T cell responses. Administration of anti-CD8 mAb to the activation of alloreactive CD8+ T cells in-vivo, in a human-mouse model cells population prevented the development of graft-versus host disease induced by injection of allogeneic CD8⁺ T cells. Thus, anti-CD8 antibody therapy might prevent CD8+ T cells-mediated graft rejection, without perturbing protective anti-viral responses and might therefore represent a significant progress over current immunosuppressive treatments. This application demonstrated that CD8 ligation results in an inhibition of T cell priming and the generation of regulatory T cells.

The present inventors have demonstrated that LCs are extremely efficient at priming naïve CD8 T cells compared to Interstitial DCs, whereas both mDCs subsets were equally efficient at inducing a secondary response. The current study was performed to analyze the parameters which might explain the superior functions of LCs in inducing CD8+T cell priming. It is demonstrated herein that CD8 ligation results not only in the inhibition of T cell priming but also triggers the generation of regulatory T cells.

DCs Purification and Culture. CD34-derived DCs were generated by culturing G-CSF mobilized CD34-HPC at 0.5×10⁶/ml in 25 cm² flask in Yssel's media (Irvine Scientific, CA or Gemini BioProducts) containing 5% autologous serum, 50 μM 2-β-mercaptoethanol, 1% L-glutamine, 1% penicillin/streptomycin, and the cytokines; GM-CSF (50 ng/ml; Immunex Corp.), FLT3-L (100 ng/ml; R&D), and TNF-α (10 ng/ml; R&D). Cultures were incubated at 37° C. with 5% CO₂ in a humidified environment, Cells were transferred to fresh medium supplemented with cytokines at the day 5 of culture, and harvested on day 9 or 10. CD1a⁺CD14⁻-LCs and CD1a⁻CD14⁺-intDCs were sorted. Purity was routinely 95-99%.

IFN-derived DC (IFN-DC) were generated by culturing CD 14⁺ monocytes (purity >90%) (1×10⁶ cells/ml) in Cellgenix media (Cellgenix) supplemented with 1% penicillin/streptomycin, and 100 ng/ml GM-CSF (Berlex) and 500 U/ml IFN-α-2b (Schering Corp) at 37° C. and 5% CO₂, fresh medium and cytokines were added on day 1, and the DCs were harvested on day 3.

LCs and dermal IntDCs were purified from normal human skin specimens. Specimens were incubated in the bacterial protease dispase type 2 (Roche) Antibiotic/Antimycotic (Gibco) for 18 h at 4° C., and then for 2 h at 37° C. Epidermal and dermal sheets were then separated, cut into small pieces (˜1-10 mm) and placed in RPMI 1640 (Gibco) supplemented with 10% fetal bovine serum (FBS). After 2 days, the cells that migrated into the medium were collected and further enriched using a Ficoll-diatrizoate gradient, 1.077 g/dl (LSM—Lymphocyte Separation Medium, MP Biomedicals). DCs were purified by cell sorting after staining with anti-CD1a FITC (OKT6; DAKO) and anti-CD14-APC (LeuM3; Invitrogen) mAbs.

T cell isolation. Cells were isolated from frozen PBMCs obtained by leukapheresis from adult volunteer donors. Naïve CD8⁻ T cells were sorted as CD45RA⁺CCR7⁺HLA-DR⁻CD8⁺ cells, following CD4⁻, CD56⁻, CD16⁻ and CD19⁻ magnetic cell depletion (Miltenyi). Naïve CD4⁺ T cells were obtained in the same manner, except that CD8 T cells were depleted and resulting cells were sorted as CD4⁺CCR7⁺CD45RA⁻CD4⁻CD16⁻CD19⁻CD56⁻. For recall responses, CD8⁺ T cells were positively selected from an enriched population.

DC/CD8 T Cell Cocultures. Autologous CD8⁺ T cells—DCs coculture. For primary response assessments, naïve CD8⁺ T cells (1×10⁶ cells/well) were stimulated with autologous mDCs (5×10⁴ cells/well) that were preincubated for 3 h with the HLA-A201-restricted MART-1 (MART-1_{M26-35}, ELAGIGILTV) or gp100 (gp100_{M209-217}, IMDQVPFSV) peptide (3 μM). Cells were cultured for 9 days in 24-well plates in Yssel's complete medium supplemented with 10 U/ml IL-7 (R&D) and 100 ng/ml CD40L (R&D). IL-2 (R&D) was added at 10 U/ml at day 3; anti-CD8 or isotype matched control was added on day 0, unless otherwise indicated.

Expansion of peptide-specific CD8⁺ T cells was determined by counting the number of cells binding peptide/HLA-A201 tetramers (Beckman Coulter) at the end of the culture period. For the assessment of recall responses, total CD8⁻ T cells (1×10⁶ cells/ml) were stimulated with autologous (5×10⁵ cells/ml) mDC subsets loaded with HLA-A201-restricted Flu-MP peptide (GILGFVFTL). In the presence of anti-CD8 or isotype matched control. The frequency of Flu-MP-specific CD8⁺ T cells was determined by using Flu-MP/HLA-A201 tetramer.

Allogeneic CD8 T cell cultures. Allogeneic proliferation of naïve CD8⁺ T cells was assessed by [H³]-thymidine incorporation, or CFSE dilution. Naïve T cells (1×10⁵ cells/well) were cultured in round-bottomed 96-well plates in Yssel's medium supplemented with 10% heat-inactivated pooled AB human serum (Yssel's complete medium) IL-7 and IL-2 (10 IU/ml R&D), to which 2.5×10⁴ (unless otherwise indicated) allogeneic mDC subsets were added. CD40L was used to activate the DCs. After 5 days, cells were pulsed for 18 hours with 1 μCi [H³]-thymidine and the incorporation of the tracer determined as a measure of ongoing proliferation.

For assessment of proliferation by CFSE dilution, cells were labelled with 0.5 μM CFSE according to the manufacturer procedure. After 7 d, cells were harvested and the level of proliferation was analyzed by flow cytometry. In addition, the quality of the primed CD8⁺ T cells was assessed as described below.

Where indicated, blocking antibody against CD8 (clone RPA-T8, OKT6, BD, or T8 Beckman Coulter) or isotype control antibody was added to the coculture.

For secondary allogeneic CD8+ T Cell Culture, 5×10⁴ naive CD8 T cells were cultured with 2.5×10³ CD40 ligand-activated DCs in 96-well round-bottom plates with the addition of IL-7 and IL-2. After 6 d, cells were restimulated with DCs from the same donor used in the primary culture. Anti-CD8 antibody or isotype matched control was added to the culture for 3 day after which time the cellular proliferation was assessed by [³H]thymidine incorporation.

Cytokines production. For CD8+ T cells cytokine production assessment, the proliferated CD8⁺ T cells (FSC^{high}CD11c⁻ or CFSE^{low}CD11c⁻) were isolated on day 7 by cell sorting from a primary allogeneic culture and restimulated overnight with anti-CD3 and anti-CD28 coated microbeads. Cytokines in the supernatant were measured by multiplex bead-based cytokine assay.

CD8⁺ T Suppressor assay. For CD8⁺ T Suppressor function Assay, the proliferated CD8⁺ T cells (FSC^{high}CD11c⁻ or CFSE^{low}CD11c⁻) were isolated on day 7 by cell sorting from a primary allogeneic culture and added at graded numbers to a coculture of 5×10⁴ naive CD8⁺ T cells and CD40L-activated 2.5×10³ allogeneic DCs (LCs). 1 μ/Ci of [³H]thymidine was added to each well After 5 d of culturing, and cellular incorporation was determined after 18 h.

T-cell protein and gene analysis. For effector molecules staining, primed CD8⁺ T cells were fixed and permeabilized and stained with PE-labeled anti-GranzymeA, GranzymeB and perforin (BD Biosciences).

For CD8⁺ T cells phenotype analysis, cells were stained for surface expression of CD25 (M-A251), CD28 (CD28.2), CCR7, CD103 (Ber-ACT8) all from BD biosciences.

For microarray gene analysis, the proliferating CD8 T cells (CFSE⁻) from a primary allogeneic culture were sorted and re-stimulation with anti-CD3 and anti-CD28 coated microbeads . . .

Evaluation of Anti-CD8 treatment against Graft vs. host disease in vivo. Mobilized peripheral blood (MPB) CD34⁺ cells (3-6×10⁶ MPB CD34⁻ cells per animal) were infused intravenously into separate experimental cohorts of sublethally irradiated (300 centigrays by ¹³⁷Cs γ-irradiation) NOD/SCID mice as previously described 10-12 weeks after transplantation, mice were injected subcutaneously with 10M sorted naïve CD8⁺ T cells from an allogeneic donor. Mice were treated with an IgG1 control mAb or anti-CD8 mAb (RPA-T8 BD biosciences, 0.75 mg on day 0 and 0.25 mg on day 3) subcutaneously. In one out of two experiments anti-CD40 monoclonal antibody (MAB89, Schering-Plough) was injected intra-peritoneally at the day of the allogeneic transplantation to activate the DCs.

Mice were observed daily for survival and clinical signs of GVHD, as manifested by diarrhea, weight loss and ruffled skin. When symptoms appeared mice were harvested. Human CD8⁺ T cells were analyzed by flow cytometry.

In vitro priming of naïve CD8⁻T cells with in vitro generated LCs. HLA-A201⁺LCs and IntDCs were generated in-vitro by culturing for nine to ten days CD34⁺ HPCs in the presence of GM-CSF, Flt3-L and TNFα. Cells were sorted into CD1a⁺CD14⁻LCs (LCs) and CD1a⁻CD14⁺ IntDCs (IntDCs). For primary response, DCs subsets loaded with 3 μM HLA-A201-restricted melanoma peptide MART-1 (26-35) were cultured with autologous naïve CD8⁺ T cells for nine to ten days. The frequency of the antigen specific CD8⁺ T cells at the end of the culture was measured using specific peptide-MHC tetramer.

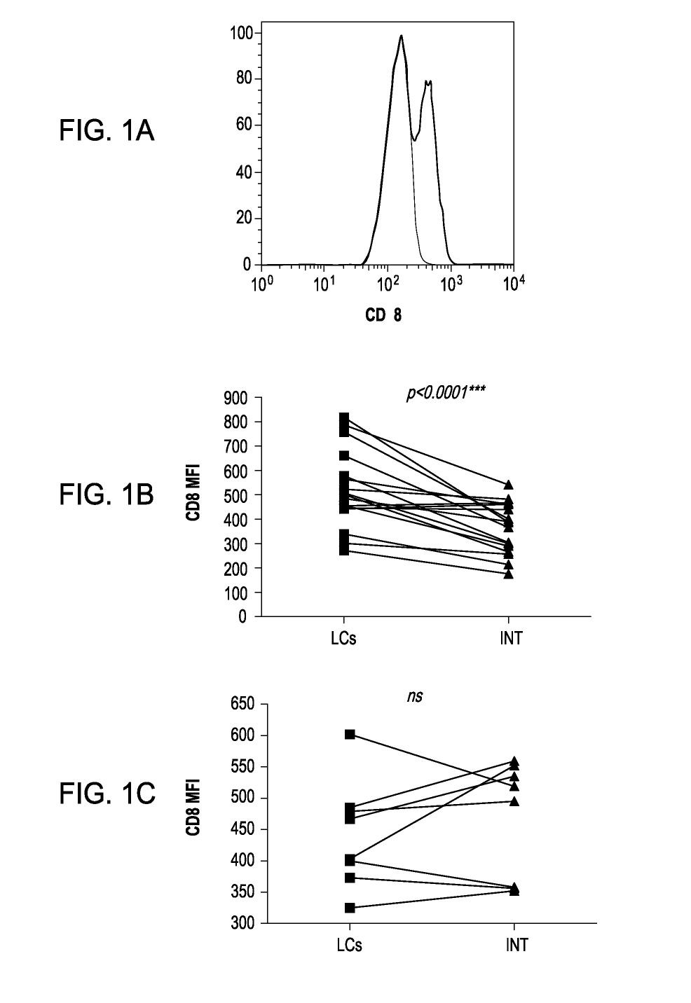

As shown in FIG. 1a and FIG. 1b, naïve CD8⁺ T cells primed by LCs upregulate surface CD8 expression when compared with IntDCs-primed CD8⁺ T cells. For memory response, DCs subsets were loaded with 1 μM of the HLA-A201 restricted influenza matrix peptide M1. DCs were cultured with sorted autologous memory CD8⁺ T cells. In contrast, both subsets are equally efficient at inducing a secondary response to a viral antigen, and the CD8⁺ T cells activated by either subset express equal levels of surface CD8 (FIG. 1c).

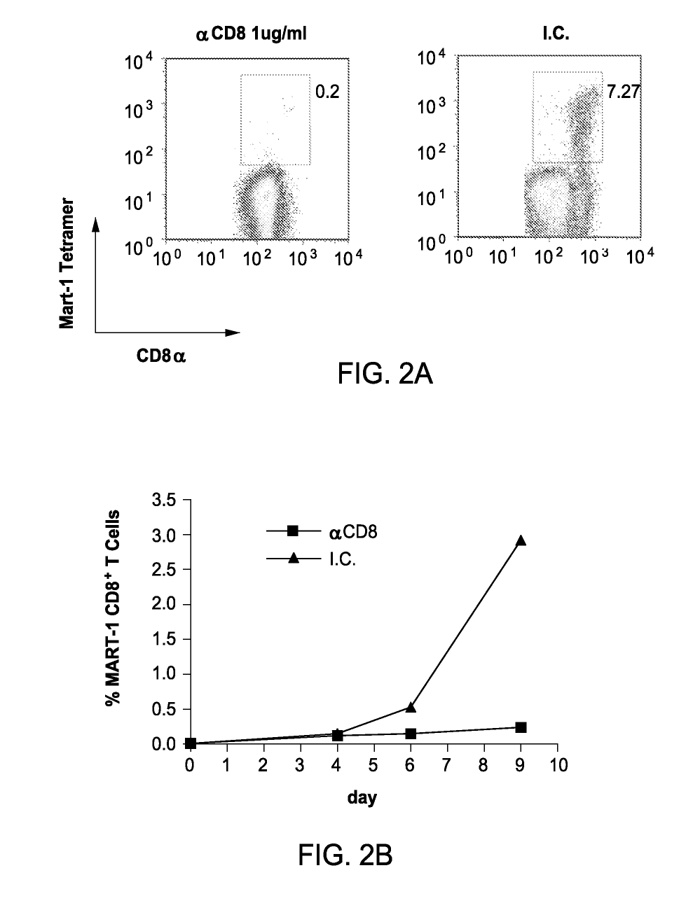

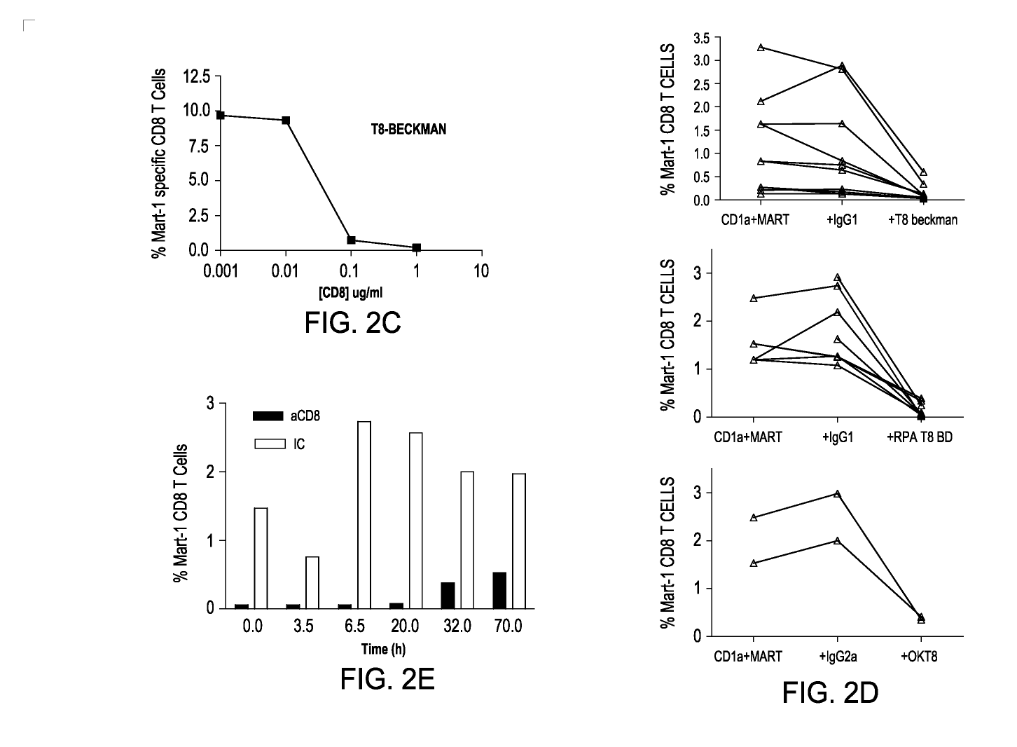

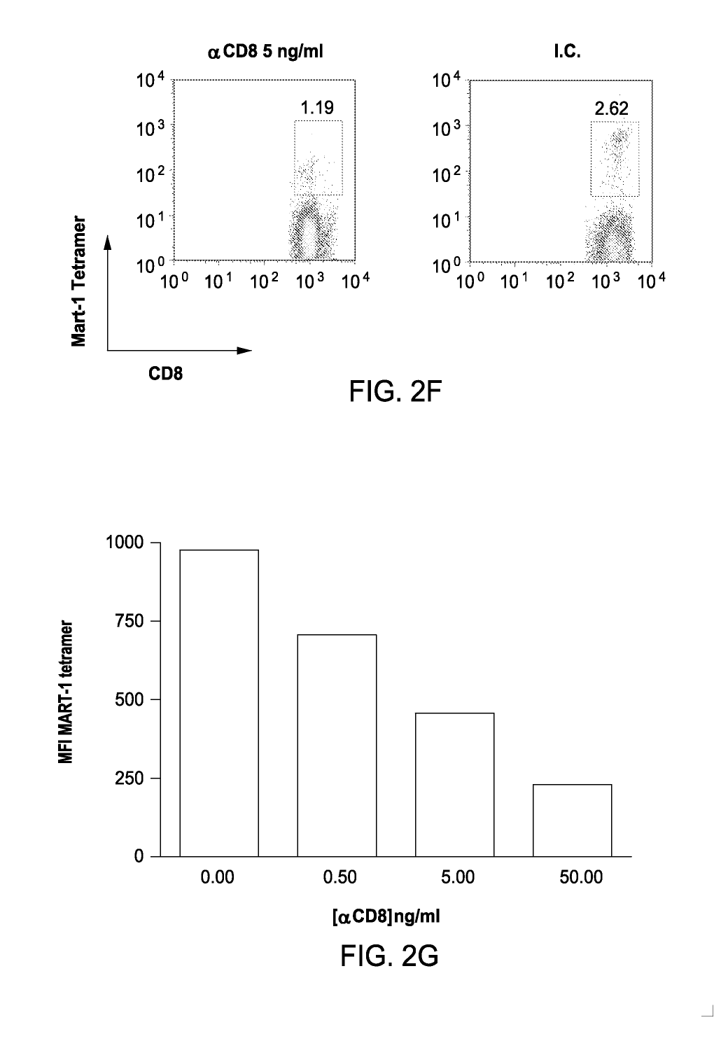

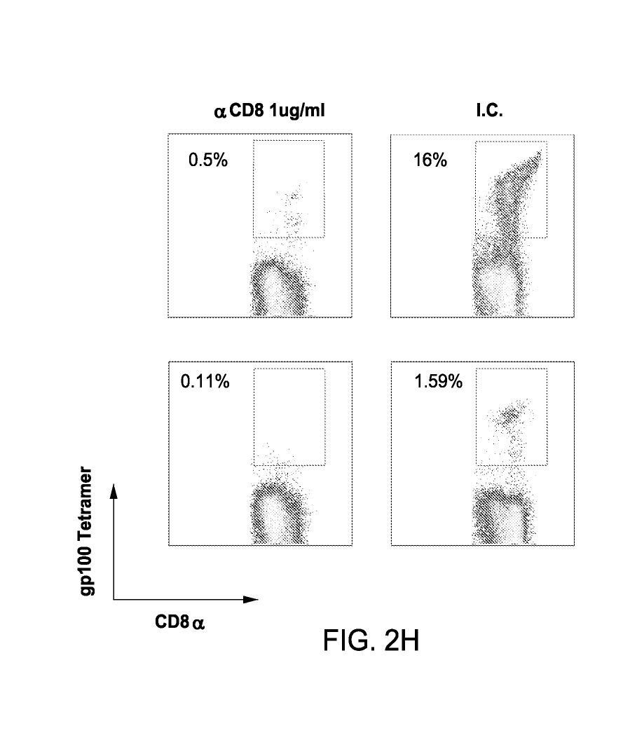

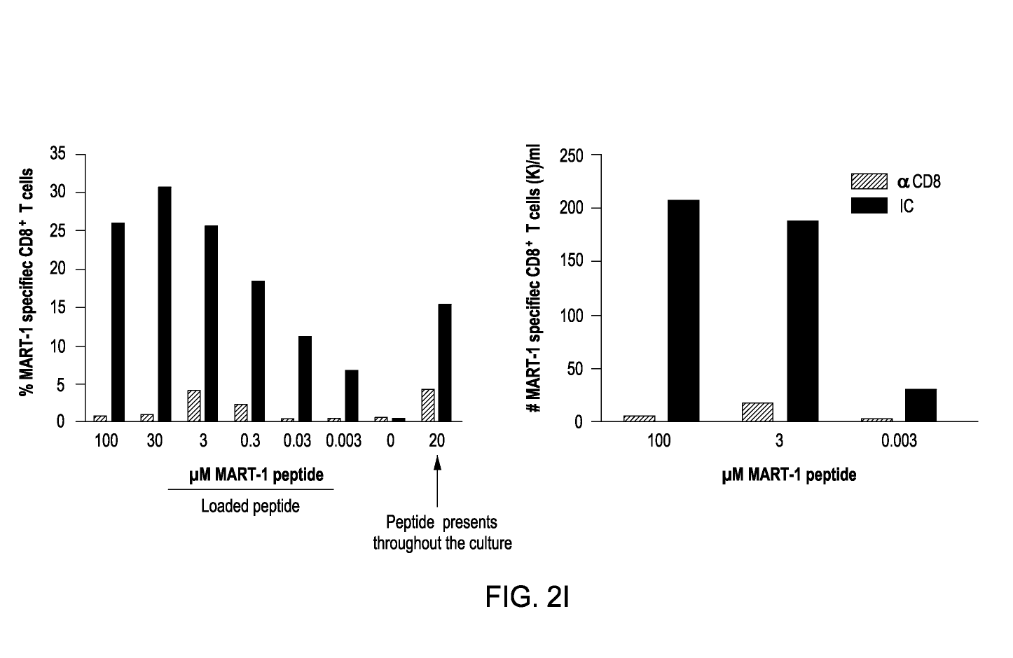

Anti-CD8 antibody prevents priming of antigen specific CD8⁺T cells. Addition of the anti-CD8 mAb RPA-T8 efficiently blocked the expansion of MART-1 specific CD8⁺ T cells by MART-1-pulsed LCs (FIG. 2a). A kinetic analysis indicates that very little antigen-specific CD8⁻T cells proliferation is observed between day one and day nine when the anti-CD8 mAb is added to the culture (FIG. 2b). The inhibition of CD8⁺ T cell priming was very effective as 0.1 μg/ml of antibody resulting in near complete inhibition of the expansion of antigen specific CD8⁺T cells and the 50% Inhibitory Concentration (IC₅₀) was in the range of 50-500 ng/ml (FIG. 2c). Three out of three tested anti-CD8 antibodies (T8, RPA-T8 and OKT8) inhibited T cell priming (FIG. 2d).

Delaying addition of anti-CD8 mAb to cultures until hour seventy still resulted in a 75% inhibition of melanoma specific CD8⁺ T cell priming (FIG. 2e). Culturing MART-1 peptide-loaded LCs and naïve CD8⁺ T cells in the presence of low concentration of anti-CD8, resulted in decreased number of MART-1 specific CD8⁺ T cells compared to primary control cultures (FIG. 2f) furthermore CD8⁺ T cells exposed to anti-CD8 mAb showed lower MART-1 MHC-tetramer intensity staining when compared to those exposed to the control antibody. The more anti-CD8 mAb was added to cultures, the less tetramer intensity binding was observed on antigen-specific T cells (FIG. 2g).

The anti-CD8 mAb was also able to block the priming of MART-1 and gp100-specific CD8⁺ T cells induced by DCs generated by culturing monocytes with GM-CSF and IFN (IFN-DCs) (FIG. 2h), indicating that the inhibitory effect is neither dependent on the source of DCs nor on the antigen selected for priming. In addition, anti-CD8 mAb was able to block the priming even when high concentration of peptide was loaded on the DCs, or when the antigen was present throughout the culture (FIG. 2i). Taken together these data demonstrate that blocking CD8 prevents DCs-induced priming of high-avidity antigen-specific naïve T cells.

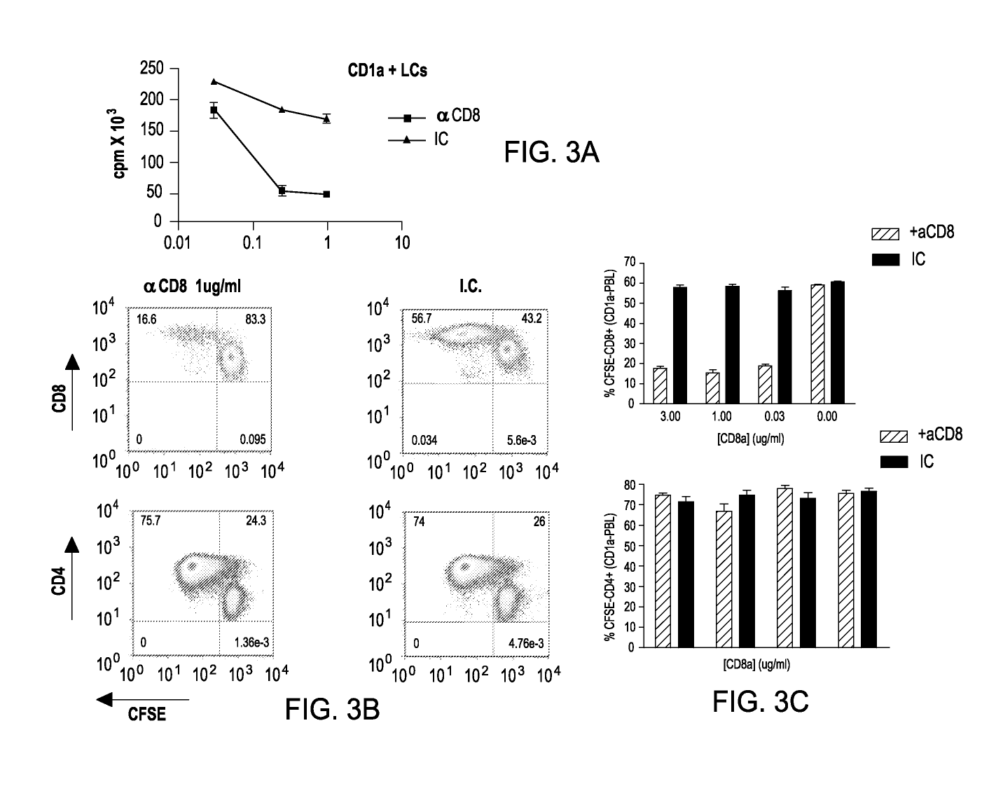

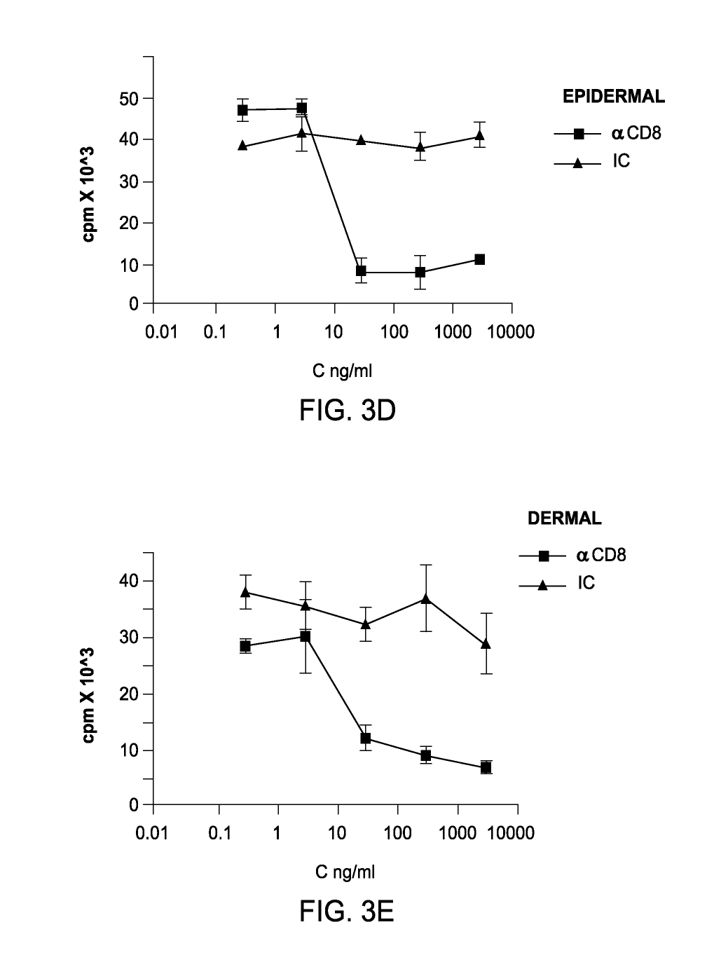

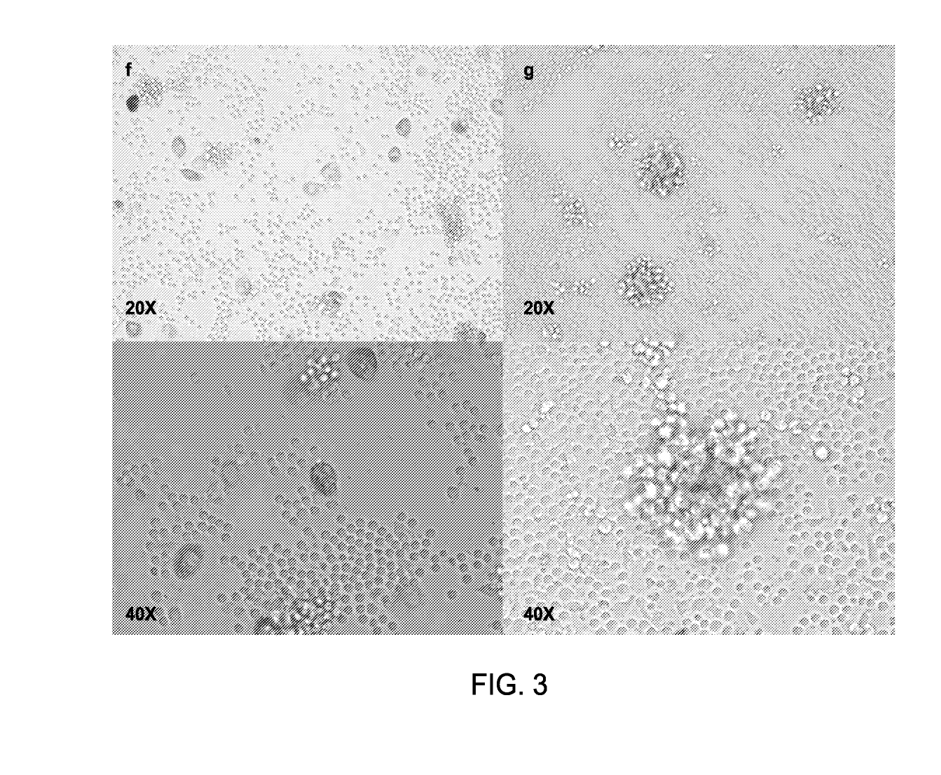

Anti-CD8 antibody inhibits DCs mediated alloproliferation of CD8 T cells. Anti-CD8 mAb or isotype control was added to cultures of naïve CD8⁺ T cells together with graded number of in-vitro generated allogeneic LCs. As shown in FIG. 3a using an [³H]thymidine incorporation assay, the LCs induced proliferation of allogeneic naïve CD8⁺ T cells, was inhibited by the anti-CD8 mAb. CFSE dilution assays performed on cocultures of LCs with allogeneic naïve CD4⁺ and CD8⁺ T cells confirmed the inhibition of CD8⁺ T cell proliferation (FIG. 3b upper panel). It further revealed that the proliferation of allogeneic CD4⁻ T cells was not affected by the anti-CD8 antibody (FIG. 3b lower panel). Indeed, while CD8⁺ T cell proliferation was inhibited at 30 ng/ml (FIG. 3c upper panel), CD4⁺ T cells showed no decreased proliferation for any concentration of anti-CD8 mAb used (0-3 μg/ml) (FIG. 3c lower panel). The vigorous proliferation of allogeneic T cells induced by dermal DCs or LCs isolated from human skin was also blocked by anti-CD8 mAb (FIGS. 3d and e). In the presence of anti-CD8 mAb only few, scattered, small clusters were formed between CD8⁺T cells and DCs (FIG. 3f). However, in cultures with no anti-CD8 mAb, vigorous proliferation was associated with many large clusters of DCs and CD8⁺ T cells (FIG. 3g). Thus, anti-CD8 antibody can inhibit DCs-mediated priming of allogeneic CD8⁺ T cells.

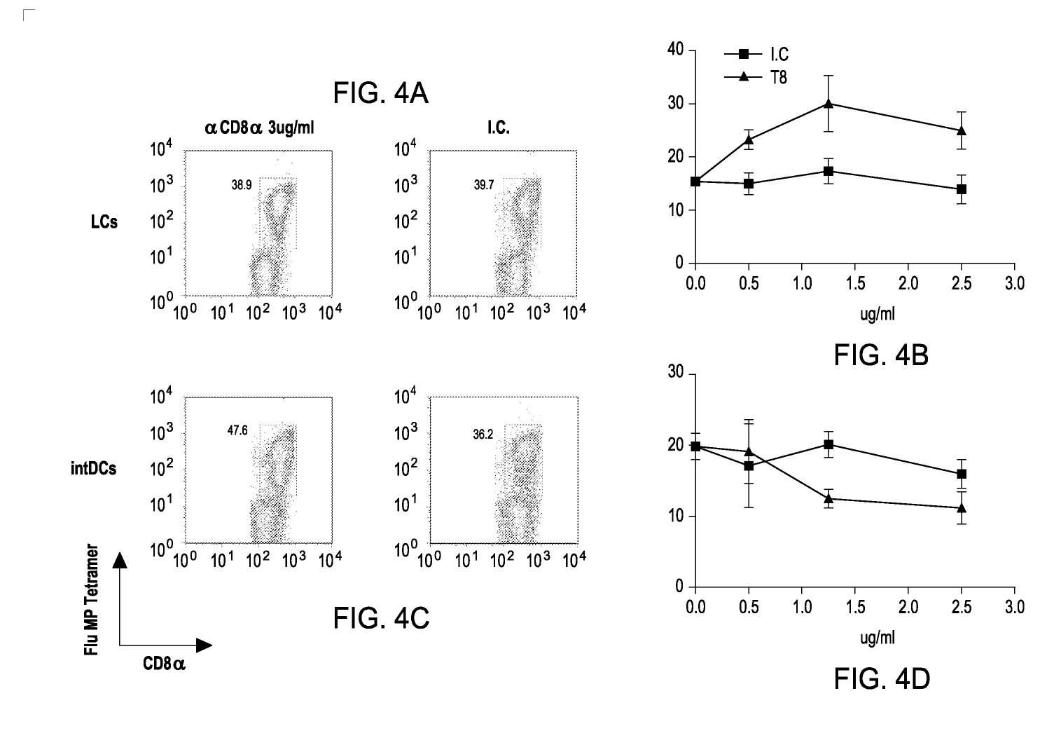

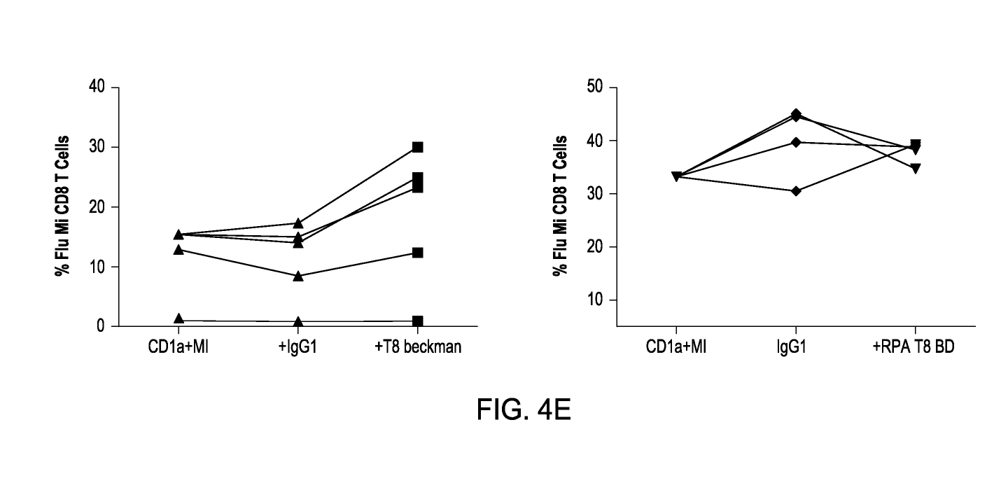

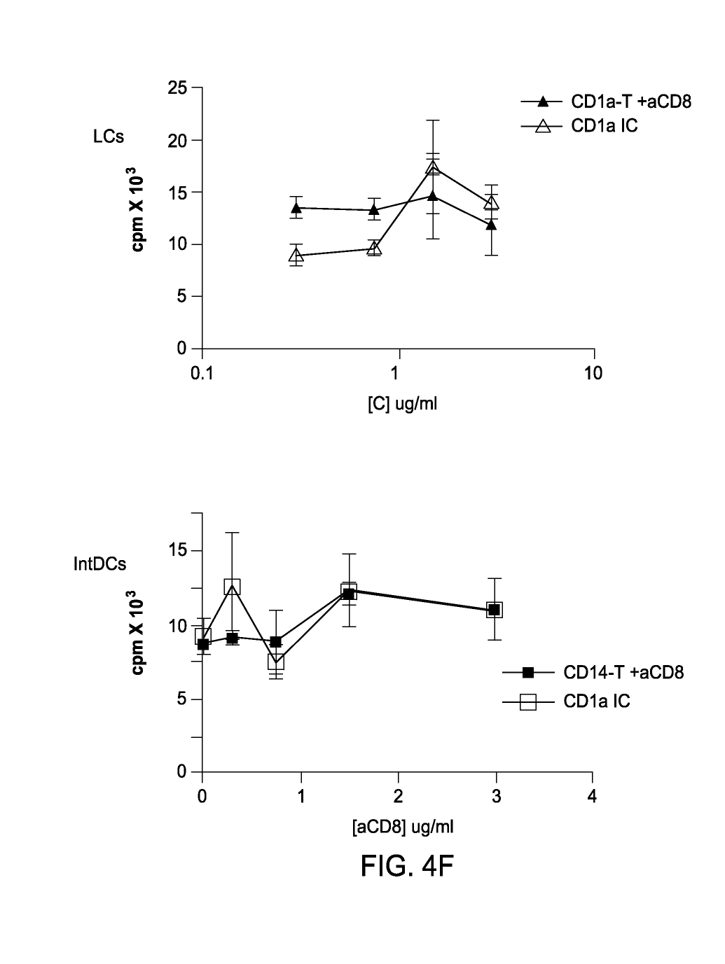

Anti-CD8 does not block secondary response against autologous or allogenic antigens. To test whether memory CD8⁺ T cell responses would also be inhibited by anti CD8 mAb, HLA-A2⁺ LCs or IntDCs, loaded with the immunodominant HLA-A2 binding influenza matrix protein M1 peptide (57-68), were cultured with CD8⁺ T cells with the anti-CD8 mAb and its relevant control. For either DC subset, the number of antigen specific CD8⁺ T cells, as measured by tetramer staining, was comparable with anti-CD8 mAb or isotype control (FIGS. 4a and c). No inhibition was detected even with concentration of anti-CD8 mAb as high as 2.5 μg/ml (FIGS. 4b and d). The two other anti-CD8 mAbs tested (T8 Beckman, RPA-T8 BD) did not inhibit the flu peptide-induced activation of memory cells (FIG. 4e).

To demonstrate whether memory allogeneic CD8⁺ T cell responses would be affected by anti-CD8 mAb, naïve CD8⁺ T cells were primed by allogenic LCs or IntDCs for seven days, and the T cells were restimulated for three days. As shown in FIG. 4f, the anti-CD8 mAb was not able to inhibit CD8⁺ T cell restimulation with the original alloantigen using either, LCs or IntDCs. Thus, these data demonstrate that memory CD8⁺ T cell responses are CD8-independent.

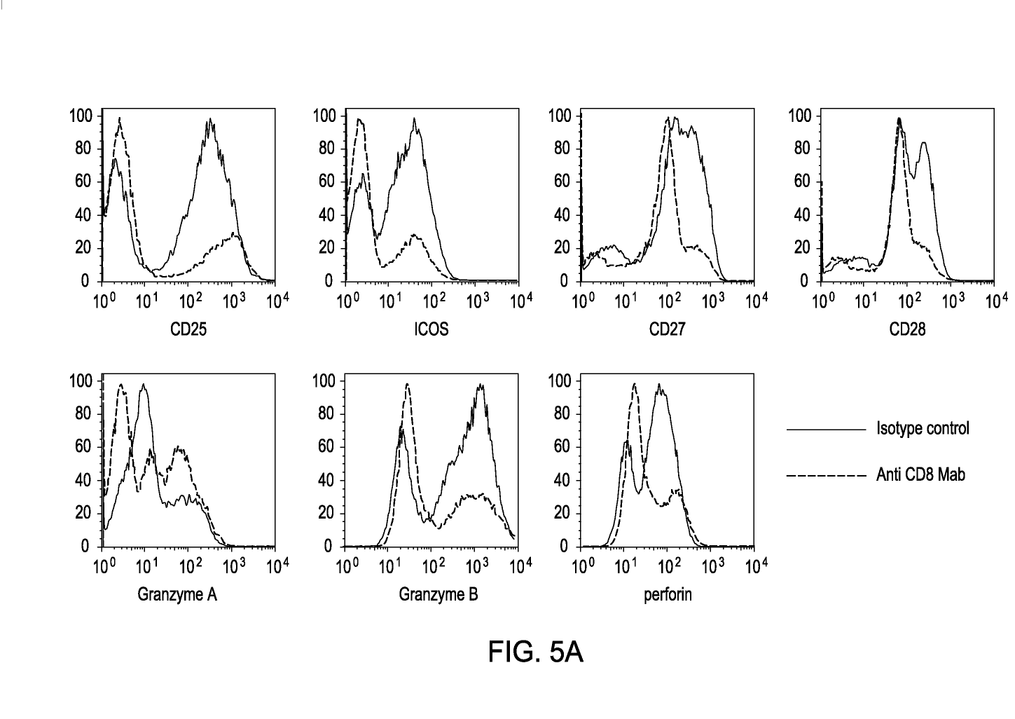

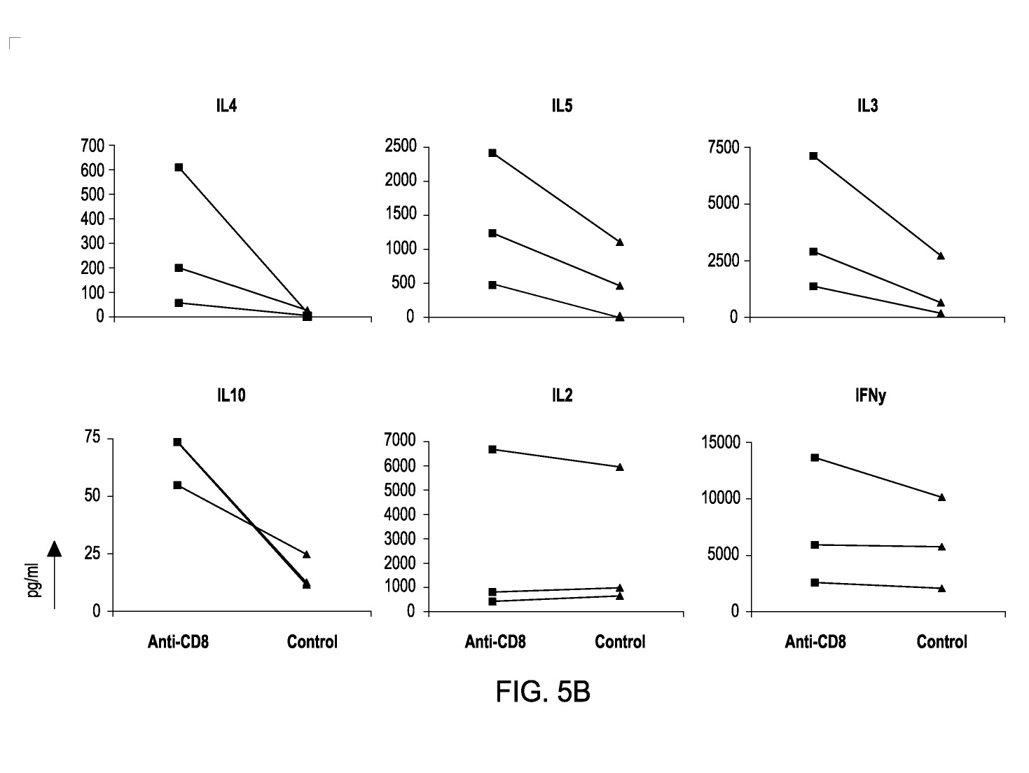

Priming CD8⁺T cells with Anti-CD8 mAb yields Type 2 T cells with low levels of cytolytic molecules. As shown in FIG. 5, CD8⁻ T cells that were exposed to anti-CD8 mAb during priming with allogeneic DCs express lower levels of CD25, ICOS, CD27, CD28 and lower intracellular expression of granzymes A and B and perforin (FIG. 5a). CD8⁺ T cells primed with LCs and isotype control for seven days produced, following restimulation with anti-CD3 plus anti-CD28 for 24h, IFN-γ (2,000-6,000 pg/ml) and IL-2 (1,000-6000 pg/ml), and low levels IL-4, IL-5, IL-13 and IL-10. CD8⁺ T cells primed with LCs and anti-CD8 secreted the same amounts of IFN-γ and IL-2 but high amounts of IL-4 (100-600 pg/ml), IL-5 (500-2500 pg/ml), IL-13 (1000-7000 pg/ml) and IL-10 (70-100 pg/ml) (FIG. 5b).

Collectively, the data indicate that anti-CD8 mAb alters the phenotype of activated CD8⁺T cells yielding cells secreting Type 2 cytokines and expressing low levels of cytotoxic molecules.

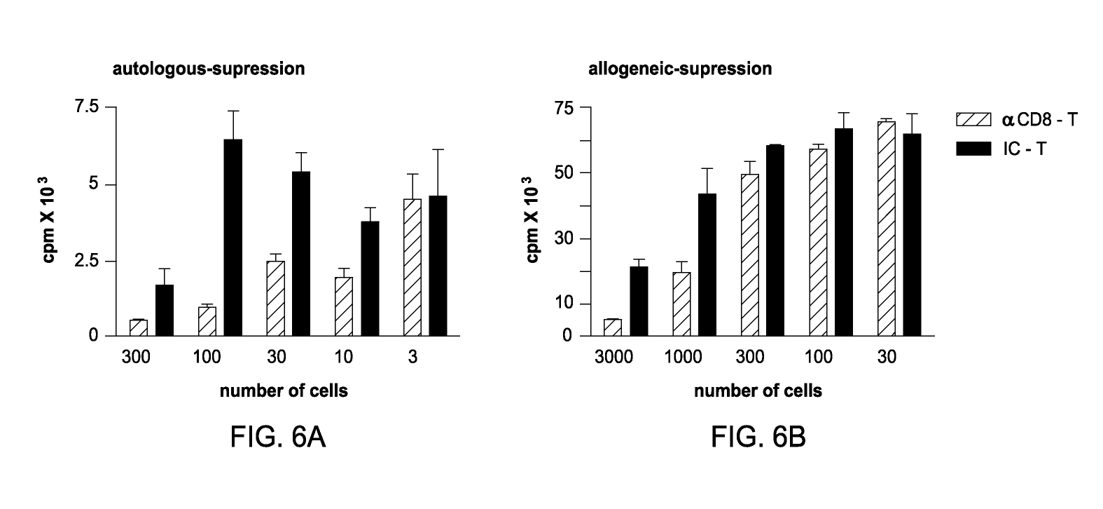

Alloreactive CD8⁺ T cells primed in the presence of anti-CD8 potently suppress naive CD8⁺ T cell responses. To determine whether CD8⁺ T cells primed in the presence of anti-CD8 mAb show suppressor functions, CFSE-labeled naïve CD8⁺T cells (donor A) were cultured with allogeneic LCs (donor B) with anti-CD8 mAb or isotype matched control for seven days. Activated CD8⁺ T cells (CFSE-CD11c-) were sorted and added at graded numbers (3-300) into a coculture of 50,000 autologous naive CD8⁺ T cells from donor A with 2500 allogeneic LCs from donor B. CD8⁺ T cells primed with anti-CD8 mAb strongly inhibited the proliferation of naive CD8⁺T cells to allogeneic LCs in a dose-dependent fashion, with as little as 100 cells suppressing the alloreaction by around 80% and ten cells blocking by 50%. However, CD8⁺ T cells primed with isotype control showed no inhibition (FIG. 6a). The inhibition was particularly striking when the anti-CD8 mAb treated CD8⁺T cells were given their allospecific DCs, as the suppression was less intense with DCs from donor C (FIG. 6b).

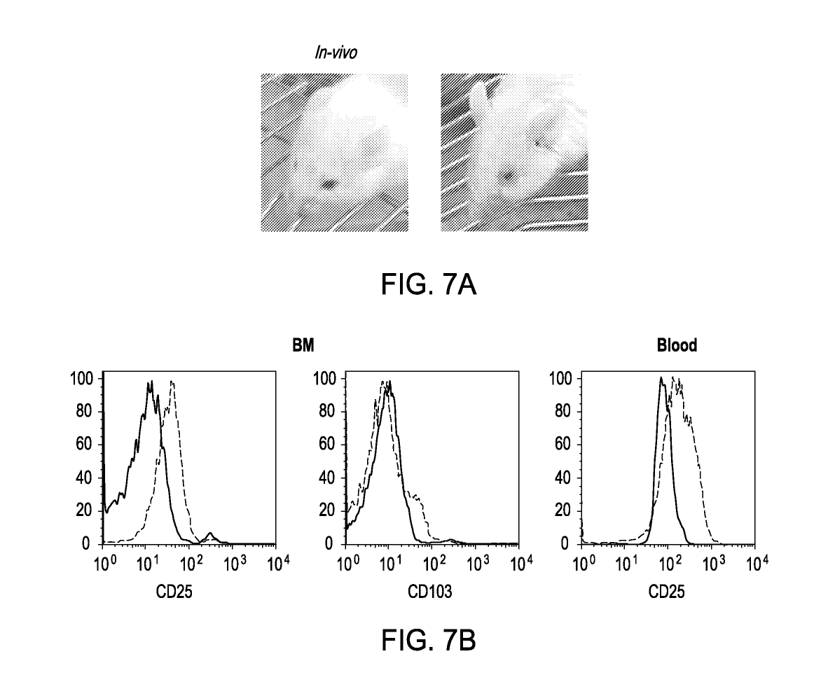

Anti-CD8 mAb inhibits allogeneic CD8⁺ T cell activation and graft-versus-host disease in-vivo. The strong inhibition of CD8⁺ T cell priming observed in vitro with anti-CD8 antibodies led us to test whether this would also happen in vivo in immunodeficient NOD-SCID mice grafted with human CD34⁺HPCs which differentiate into pDCs, mDCs and B cells but not T cells. These humanized mice were adoptively transferred subcutaneously with 20×10⁶ purified CD8⁺ T cells from an allogeneic donor with 0.75 mg of either the anti-CD8 mAb or an isotype-matched control antibody. An additional 0.25 mg of antibody was injected on day three. In one of the two experiments, anti-CD40 (MAB89, Schering Plough, 100 μg) was injected intraperitoneal in for DCs activation. Mice were examined regularly for sign of sickness. At ten weeks post CD8⁻ T cells transfer, mice receiving the isotype-matched control antibody developed clinical symptoms of chronic graft-versus-host disease, with rashes around the eyes, weight loss and weakness (FIG. 7a). Treatment with anti-CD8 antibody, however, completely inhibited both the activation and expansion of pathogenic T cells and the development of clinical symptoms (FIG. 7). CD8⁺ T cells from the bone marrow of isotype control treated mice upregulated CD103 whereas mice treated with anti-CD8 mAb did not (FIG. 7b).

Collectively these data indicate that anti-CD8 mAb therapy is efficient in preventing allogeneic primary activation of CD8⁺ T cells, which mediate graft-versus-host disease in immunodeficient mice carrying a human immune system.

The current study was performed to understand why LCs are more potent than Interstitial DCs at priming naïve CD8⁺ T cells while both mDCs subsets were equally efficient at inducing a secondary CD8⁺ T cell response. Several conclusions were drawn from results of adding anti-CD8 mAbs to cocultures of naïve CD8⁺ T cells and DCs. First priming of naïve CD8⁺ T cells was found to be profoundly inhibited at very low concentration of antibody while activation of memory cells was not affected even at high concentration of antibody. Second the residual proliferating cells differentiated along a suppressor pathway rather than an effector pathway.

These data demonstrated that the entire tested anti-CD8 monoclonal antibodies block, at very low concentration, DCs-mediated proliferation of CD8⁺ T cells against antigen presented in the context of autologous or allogeneic MHC in vitro. However, recall responses against viral or allogeneic antigens were not inhibited by anti-CD8 mAbs. These data are in line with an earlier report on in vitro studies with mouse lymphocytes showing that anti-CD8 antibodies can block the proliferation of naïve CD8⁺ T cells but not that of effector and memory cells⁶. Anti-CD8 also blocked activation of alloreactive naïve CD8⁺ T cells was also observed in-vivo in a humanized mouse model resulting in the ablation of graft versus host response. Perhaps the most striking observation is that addition of the anti-CD8 antibodies qualitatively modified the type of response from an effector response to a suppressor one. The generated suppressor cells express a unique phenotype with decreased expression of Granzyme A and B and perforin and low CD28. Furthermore, these cells express an altered phenotype pattern with increased expression of type 2 cytokines (IL-4, IL-5 and IL-13) and that of IL-10. In addition, these cells express potent suppression capacity as 100 of these cells can block 80% of an alloreaction particularly when activated by cognate APCs. Interestingly, this phenotype is comparable with the phenotype of CD8⁺ T cells cultured over CD14⁺IntDCs as we reported elsewhere.

These observations are of clinical significance as T cells are the primary mediators of allograft rejection^{11,12}. Much effort has been directed at designing therapeutics that specifically block the initial activation of T cells in allograft recipients. Both CD4⁺ T cells-dependent and CD8⁺ T cells-dependent pathways have been demonstrated to initiate allograft rejection. While immunoregulation strategies such as Rapamycin¹³, Cyclosporine¹⁴, anti-CD4 mAb¹⁵, anti-CD154 mAb¹⁶ and CTLA4-Ig¹⁷ are very effective at suppressing the CD4-dependent immune activation, the CD8-dependent pathway of rejection has been demonstrated in studies to be resistant. Resistance of CD8⁺ T cells to suppression by calcineurin inhibitors has also been correlated with an increased incidence of acute allograft rejection in clinical studies¹⁸. This is in line with the different costimulatory requirements of CD4⁺ and CD8⁺ T cells observed in vivo. CD8-dependent allograft rejection is dependent upon CD40/CD154 costimulation and independently of the CD28/B7 costimulatory pathway¹⁷.

First generation anti-CD3 mAbs block the initial activation of T cells in allograft recipients resulting in immunosuppression which, as with most other immunosuppressive treatments, is associated with severe viral infections, such as CMV. Thus the observation that anti-CD8 blocks effector cell priming while leaving virus specific memory responses intact might prevent the generation of alloreactive CD8⁺ T cells that attack a graft while leaving anti-viral secondary responses intact.

Up-regulation of CD103 by CD8⁺ T cells at the graft site has been closely linked to the ability of CD8⁺ T cells to mediate allograft damage¹⁹. The epithelial cell-specific integrin, CD103 (α_{E} integrin), defines a novel subset of alloreactive CD8⁺ CTL²⁰. Activation of the (CD4-independent) CD8-dependent pathway of allograft rejection elicits a vigorous immune response, which is highly resistant to immunoregulation. An intense focal infiltration of mainly CD8⁺CTLA4⁺ T lymphocytes during kidney rejection has been described in patients. This suggests that CD8⁺ T cells could escape from immunosuppression and participate in the rejection process. Control of both CD4 and CD8 responses maybe necessary to promote tolerance and long term survival²¹. CD8 therapy can also be beneficial in preventing the priming of autoreactive CD8⁺ T cells in autoimmune diseases such as lupus or diabetes.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Banchereau, J. & Steinman, R. M. Dendritic cells and the control of immunity. Nature 392, 245-52 (1998).

2. Caux, C. et al. CD34+ hematopoietic progenitors from human cord blood differentiate along two independent dendritic cell pathways in response to GM-CSF+TNF alpha. J Exp Med 184, 695-706 (1996).

3. Zamoyska, R. The CD8 coreceptor revisited: one chain good, two chains better. Immunity 1, 243-6 (1994).

4. Veillette, A., Bookman, M. A., Horak, E. M. & Bolen, J. B. The CD4 and CD8 T cell surface antigens are associated with the internal membrane tyrosine-protein kinase p56lck. Cell 55, 301-8 (1988).

5. Chalupny, N. J., Ledbetter, J. A. & Kavathas, P. Association of CD8 with p56lck is required for early T cell signalling events. Embo J 10, 1201-7 (1991).

6. Bachmann, M. F. et al. Developmental regulation of Lck targeting to the CD8 coreceptor controls signaling in naive and memory T cells. J Exp Med 189, 1521-30 (1999).

7. Tewari, K., Walent, J., Svaren, J., Zamoyska, R. & Suresh, M. Differential requirement for Lck during primary and memory CD8+ T cell responses. Proc Natl Acad Sci USA 103, 16388-93 (2006).

8. Fung-Leung, W. P. et al. The lack of CD8 alpha cytoplasmic domain resulted in a dramatic decrease in efficiency in thymic maturation but only a moderate reduction in cytotoxic function of CD8+ T lymphocytes. Eur J Immunol 23, 2834-40 (1993).

9. Nakayama, K. et al. Requirement for CD8 beta chain in positive selection of CD8-lineage T cells. Science 263, 1131-3 (1994).

10. de la Calle-Martin, O. et al. Familial CD8 deficiency due to a mutation in the CD8 alpha gene. J Clin Invest 108, 117-23 (2001).

11. Hall, B. M. Cells mediating allograft rejection. Transplantation 51, 1141-51 (1991).

12. Rosenberg, A. S. & Singer, A. Cellular basis of skin allograft rejection: an in vivo model of immune-mediated tissue destruction. Annu Rev Immunol 10, 333-58 (1992).

13. Slavik, J. M., Lim, D. G., Burakoff, S. J. & Hafler, D. A. Rapamycin-resistant proliferation of CD8+ T cells correlates with p27kipl down-regulation and bcl-xL induction, and is prevented by an inhibitor of phosphoinositide 3-kinase activity. J Biol Chem 279, 910-9 (2004).

14. Boleslawski, E. et al. Defective inhibition of peripheral CD8+ T cell IL-2 production by anti-calcineurin drugs during acute liver allograft rejection. Transplantation 77, 1815-20 (2004).

15. Jones, N. D. et al. CD40-CD40 ligand-independent activation of CD8+ T cells can trigger allograft rejection. J Immunol 165, 1111-8 (2000).

16. Guo, Z. et al. CD8 T cell-mediated rejection of intestinal allografts is resistant to inhibition of the CD40/CD154 costimulatory pathway. Transplantation 71, 1351-4 (2001).

17. Newell, K. A. et al. Cutting edge: blockade of the CD28/B7 costimulatory pathway inhibits intestinal allograft rejection mediated by CD4+ but not CD8+ T cells. J Immunol 163, 2358-62 (1999).

18. Zhai, Y., Meng, L., Gao, F., Busuttil, R. W. & Kupiec-Weglinski, J. W. Allograft rejection by primed/memory CD8+ T cells is CD154 blockade resistant: therapeutic implications for sensitized transplant recipients. J Immunol 169, 4667-73 (2002).

19. Hadley, G. A., Bartlett, S. T., Via, C. S., Rostapshova, E. A. & Moainie, S. The epithelial cell-specific integrin, CD103 (alpha E integrin), defines a novel subset of alloreactive CD8+ CTL. J Immunol 159, 3748-56 (1997).

20. Feng, Y. et al. CD103 expression is required for destruction of pancreatic islet allografts by CD8(+) T cells. J Exp Med 196, 877-86 (2002).

21. Cobbold, S. P., Martin, G. & Waldmann, H. The induction of skin graft tolerance in major histocompatibility complex-mismatched or primed recipients: primed T cells can be tolerized in the periphery with anti-CD4 and anti-CD8 antibodies. Eur J Immunol 20, 2747-55 (1990).

What is claimed is:

1. A method of inducing tolerance in a subject in need thereof comprising:
contacting isolated T cells with an amount of non-depleting anti-CD8 antibody during T cell priming with an antigen effective to induce tolerogenic T cell; and
providing the subject in need or tolerance with the tolerogenic T cells.

2. The method of claim 1, wherein the anti-CD8 antibody is humanized.

3. The method of claim 1, wherein the anti-CD8 antibody is non-depleting.

4. The method of claim 1, wherein the generation of suppressor T cells is determined by determining one or more of the following phenotypes: a reduction in granzyme A, a reduction in granzyme B, a reduction of perforin, secretion of reduced amounts of IL-2, IFN-γ or both, secretion of IL-10 or a combinations thereof.

5. The method of claim 1, wherein the generation of suppressor T cells is the proliferation of suppressor T cells that secrete IL-10.

6. The method of claim 1, wherein the anti-CD8 antibody is selected from cM-T807, T8, RPA-T8, HIT8a, Leu 2, T8, and OKT8.

7. The method of claim 1, wherein the antigen is allogeneic.

8. A method to reduce transplant rejection in a transplant patient while maintaining other immune responses comprising:
treating isolated CD8⁺ T cells with an amount of anti-CD8 non-depleting, blocking antibody effective to trigger the generation of suppressor CD8⁺ T cells during priming with an antigen, wherein the suppression of the T cells is characterized by one or more of the following phenotypes: a reduction in granzyme A, a reduction in granzyme B, a reduction of perforin, secretion of reduced amounts of IL-2, IFN-γ or both, secretion of IL-10 or a combinations thereof; and
introducing the suppressor CD8⁺T cells into the transplant patient.

9. The method of claim 8, wherein the CD8⁺ T cells are incubated with isolated dendritic cells obtained from monocytes cultured with GM-CSF and IFN-α-2b (IFN-DCs).

10. The method of claim 9, wherein the dendritic cells are Langerhans cells (LCs) generated in-vitro by culturing CD34+ human peripheral cells for nine to ten days with GM-CSF, Flt3-L and TNFα.

11. The method of claim 9, wherein the dendritic cells are CD1a+CD14− LCs.

12. The method of claim 8, wherein the anti-CD8 antibody down-regulates the immune response to the engrafted organ without affecting the immune response to viruses.

13. The method of claim 8, wherein the CD8⁺ T cells treated with the anti-CD8 antibody are high-avidity, antigen-specific naïve T cells.

14. The method of claim 8, wherein the anti-CD8 antibody is selected from cM-T807, T8, RPA-T8, HIT8a, Leu 2, T8, and OKT8.

15. The method of claim 8, wherein the anti-CD8 antibody is provided in the culture at between 0.5 to 5,000 ng/ml.

16. The method of claim 8, further comprising the steps of isolating peripheral blood mononuclear cells, isolating LC precursors from the peripheral blood mononuclear cells, culturing the LC precursors with GM-CSF, Flt3-L and TNFα to make LCs, isolating T cells from peripheral blood mononuclear cells and co-culturing the LCs and the T cells in the presence of an anti-CD8 antibody under conditions that generate suppressor T cells, and reintroducing the T cells, the LCs or both into a patient prior to, in conjunction with or after transplantation.

17. The method of claim 8, further comprising the steps of isolating peripheral blood mononuclear cells from the transplant patient, isolating LCs and culturing the LCs GM-CSF, Flt3-L and TNFα, isolating T cells from the transplant patient and co-culturing the LCs and the T cells in the presence of an anti-CD8 antibody to generate suppressor T cells, and reintroducing the T cells, the LCS or both into the patient prior to, in conjunction with or after transplantation.

18. The method of claim 8, wherein the suppressor CD8⁺ T cells have an increased expression of type 2 cytokines (IL-4, IL-5 and IL-13) and IL-10.

19. A method of making suppressor T cells comprising:
isolating peripheral blood mononuclear cells, isolating Langerhans' Cell (LC) precursors from the peripheral blood mononuclear cells, culturing the LC precursors with GM-CSF, Flt3-L and TNFα to make LCs, isolating T cells from peripheral blood mononuclear cells and co-culturing the LCs and the T cells in the presence of an anti-CD8 antibody under conditions that generate suppressor T cells.

20. The method of claim 19, wherein the anti-CD8 antibody down-regulates the immune response to the engrafted organ without affecting the immune response to viruses.

21. The method of claim 19, wherein the CD8+ T cells are high-avidity antigen-specific naïve T cells.

22. The method of claim 19, wherein the Langerhans cells are CD1a+CD14− LCs.

23. The method of claim 19, wherein the CD1a+CD14− Langerhans cells are obtained by cell sorting.

24. The method of claim 19, wherein the Langerhans cells are generated in-vitro by culturing for nine to ten days CD34+HPCs with GM-CSF, Flt3-L and TNFα.

25. The method of claim 19, wherein the anti-CD8 antibody is selected from cM-T807, T8, RPA-T8, HIT8a, Leu 2, T8, and OKT8.

26. The method of claim 19, wherein the anti-CD8 antibody is provided in the culture at between 0.5 to 5,000 ng/ml.

27. A method of making suppressor T cells comprising:
isolating peripheral blood mononuclear cells, isolating monocytes from the peripheral blood mononuclear cells, culturing the monocytes with GM-CSF and IFN-α-2b to make (IFN-DCs), isolating T cells from peripheral blood mononuclear cells and co-culturing the IFN-DCs and the T cells in the presence of an anti-CD8 antibody under conditions that generate suppressor T cells as measured by a reduction in granzyme A, a reduction in granzyme B, a reduction of perforin, secretion of reduced amounts of IL-2, IFN-γ or both, secretion of IL-10 or a combinations thereof.

28. A method for affecting an immune response, comprising, administering a composition comprising suppressor T cells made by isolating peripheral blood mononuclear cells, isolating LC precursors from the peripheral blood mononuclear cells, culturing the LC precursors with GM-CSF, Flt3-L and TNFα to make LCs, isolating T cells from peripheral blood mononuclear cells and co-culturing the LCs and the T cells in the presence of an anti-CD8 antibody under conditions that generate the suppressor T cells.

29. A method of inhibiting rejection of a transplanted tissue in a mammal, said method comprising:
introducing a suppressor T cell made by a method comprising isolating peripheral blood mononuclear cells, isolating LC precursors from the peripheral blood mononuclear cells, culturing the LC precursors with GM-CSF, Flt3-L and TNFα to make LCs, isolating T cells from peripheral blood mononuclear cells and co-culturing the LCs and the T cells in the presence of an anti-CD8 antibody under conditions that generate the suppressor T cells.

30. A composition that reduces transplant rejection comprising an effective amount of suppressor T cells sufficient to reduce transplant rejection without eliminating other immune responses, wherein the suppressor T cells are generated from isolated peripheral blood T cells co-cultured with mature LCs in the presence of an anti-CD8 antibody under conditions that generate the suppressor T cells.

31. The composition of claim 30, wherein the anti-CD8 antibody is selected from cM-T807, T8, RPA-T8, HIT8a, Leu 2, T8, and OKT8.

32. The composition of claim 30, wherein the anti-CD8 antibody is provided in the culture at between 0.5 to 5,000 ng/ml.

33. The composition of claim 30, wherein the cells are frozen and resuspended in a medium for injection prior to use.
